SEQUENCE LISTING (14 sequences)

SEQ ID NO: 1 - DNA, 23 bp, Artificial Sequence
  Description of Artificial Sequence Synthetic primer
tctaggagccattgtgtcatgct

SEQ ID NO: 2 - DNA, 28 bp, Artificial Sequence
  Description of Artificial Sequence Synthetic primer
aatcrcacccgttagaaaatgtctttat

SEQ ID NO: 3 - DNA, 28 bp, Artificial Sequence
  Description of Artificial Sequence Synthetic probe
tggcaaaactaaatgtacagcatccaat

SEQ ID NO: 4 - DNA, 26 bp, Artificial Sequence
  Description of Artificial Sequence Synthetic primer
atgcctataacaaatgaccagaaaaa

SEQ ID NO: 5 - DNA, 28 bp, Artificial Sequence
  Description of Artificial Sequence Synthetic primer
acatatgcaaggacttcttcctttatta

SEQ ID NO: 6 - DNA, 40 bp, Artificial Sequence
  Description of Artificial Sequence Synthetic probe
tgtcaagcaatgttcagatagtaaggcaacaaagttattc

SEQ ID NO: 7 - DNA, 29 bp, Artificial Sequence
  Description of Artificial Sequence Synthetic primer
gcatatgttgtacagctacctatctatgg

SEQ ID NO: 8 - DNA, 30 bp, Artificial Sequence
  Description of Artificial Sequence Synthetic primer
tcctctatcagtccttgttaaacaaatatt

SEQ ID NO: 9 - DNA, 28 bp, Artificial Sequence
  Description of Artificial Sequence Synthetic primer
cacartaccatcctctatcagtccttgt

SEQ ID NO: 10 - DNA, 31 bp, Artificial Sequence
  Description of Artificial Sequence Synthetic probe
tgctggaaattacacacatcacctctatgca

SEQ ID NO: 11 - DNA, 25 bp, Artificial Sequence
  Description of Artificial Sequence Synthetic primer
agcttcagtcaattcaacagaagrt

SEQ ID NO: 12 - DNA, 26 bp, Artificial Sequence
  Description of Artificial Sequence Synthetic primer
aggtccaatgatattgctggtgttat

SEQ ID NO: 13 - DNA, 28 bp, Artificial Sequence
  Description of Artificial Sequence Synthetic primer
aagtccaaagatattgctggtgttattc

SEQ ID NO: 14 - DNA, 24 bp, Artificial Sequence
  Description of Artificial Sequence Synthetic probe
tgtgcggcarttttcagacaatgc

(19) United States
(12) Patent Application Publication
(10) Pub. No.: US 2010/0041019 A1
(43) Pub. Date: Feb. 18, 2010

(54) Methods of Screening for Respiratory Synctial Virus and Human Metapneumovirus

(76) Inventors: Jill Detmer, Kensington, CA (US); Sukhwinder Sangha, Fremont, CA (US); Charlene Bush-Donovan, Livermore, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/444,441

(22) PCT Filed: Oct. 2, 2007
(86) PCT No.: PCT/US07/80233

§ 371 Date: Apr. 6, 2009

Related U.S. Application Data
(60) Provisional application No. 60/828,714, filed on Oct. 9, 2006.

(51) Int. Cl. C12Q 1/70

(52) U.S. Cl. 435/5

(57) ABSTRACT

Provided are nucleic acid primers and probes for use in diagnostic assays to screen for respiratory infections, such as respiratory syncytial virus (“RSV”) and human metapneumovirus (hMPV). The primers and probes may be used to screen for RSV or hMPV in a singleplex assay or they may be used in a multiplex assay to simultaneously screen for RSV and hMPV, or RSV and/or hMPV and any of the following viruses: influenza A, and influenza B, parainfluenza viruses, adenovirus, coronavirus, and rhinoviruses.

### CROSS-REFERENCE TO RELATED APPLICATIONS

This application claims priority under 35 U.S.C. §119(e) to U.S. Provisional Application No. 60/828,714, filed on Oct. 9, 2006, the disclosure of which is incorporated herein by reference in its entirety.

### TECHNICAL FIELD

The invention relates generally to diagnostic assays to screen for respiratory infections and more specifically to nucleic acid primers and probes for use in diagnostic assays to screen for respiratory syncytial virus (“RSV”) and human metapneumovirus (“human MPV” or “hMPV”).

### BACKGROUND OF THE INVENTION

Acute respiratory infections are common in otherwise healthy infants and children. The majority of acute respiratory infections have a viral etiology. Most acute respiratory infections affect the upper respiratory tract and are mild and self-limited; however, aspiration of contaminated secretions has been implicated in the development of lower respiratory tract infections. The most common causes of acute viral respiratory infections are certain strains of adenovirus, coronavirus, rhinoviruses, influenza viruses, parainfluenza viruses, RSV, and hMPV. RSV accounts for the majority of lower respiratory tract infections and along with hMPV and influenza tends to be seasonal with the majority of cases occurring during the winter months.

RSV is a negative sense, single-stranded RNA virus of the Paramyxoviridae family, which includes common respiratory viruses such as those causing measles and mumps. RSV, which is a member of the paramyxovirus subfamily pneumovirinae, has long been recognized as a major cause of respiratory tract infection in infants and young children; however, the disease is not limited to children and is known to strike the elderly, adults with underlying cardiopulmonary disease, and immunocompromised individuals. RSV-caused pneumonia has the highest morality rate in transplant patients compared to pneumonia caused by other respiratory tract viruses.

Human MPV is also a virus of the Paramyxoviridae family that was identified in June 2001 as a cause of respiratory tract disease in Dutch children. van den Hoogen et al., NAT MED 7(6):719-724 (2001). Human MPV is closely related to the avian metapneumovirus (AMPV) subgroup C. The virus causes mild to severe bronchiolitis and pneumonia in the same populations as those that are infected with RSV. Co-infection with both RSV and hMPV is generally associated with more severe viral symptoms.

The clinical presentation and radiographic results for RSV and hMPV are very similar. RSV is most commonly diagnosed by rapid antigen or direct fluorescent antibody testing; however the accuracy of the results relies heavily on how the sample is obtained and the viral load. In pediatric patients, because viral load tends to be high, the accuracy of the antibody testing also tends to be quite high (>80%). By contrast, in adult patients, because viral shedding is much lower, the accuracy of the antibody testing tends to be quite low (˜25%). Currently, no antigen tests exist for hMPV, which has proven to be difficult to grow in culture.

Determination of the etiological agent in moderate to severe illness is often necessary for infection control purposes and to determine specific therapy. Diagnosis is often not easy to make on the basis of clinical signs and symptoms because of the similar spectrums found among various diseases. Rapid antigen testing for respiratory viruses is widely used in clinical labs; however, molecular assays have the potential to offer improvements over rapid antigen methods through increased sensitivity and specificity.

In order to be able to properly screen for RSV and hMPV, there exists a need in the art for a rapid and effective molecular diagnostic assays in order to be able to accurately test for these two pathogenic viruses.

### SUMMARY OF THE INVENTION

The present invention overcomes the need in the art by providing molecular based assays for the rapid and sensitive detection of RSV A, RSV B, and hMPV.

In one aspect of the invention, there is provided a method for detection of RSV A in a sample comprising the steps of obtaining a tissue sample from a patient; extracting nucleic acid from the sample; amplifying the nucleic acid, wherein the nucleic acid is amplified and detected with amplification primers and detection probes of SEQ ID NOS. 1, 2 and 3.

In another aspect of the invention, there is provided a method for detection of RSV B in a sample comprising the steps of obtaining a tissue sample from a patient; extracting nucleic acid from the sample; amplifying the nucleic acid, wherein the RNA is amplified and detected with amplification primers and detection probes of SEQ ID NOS. 4, 5 and 6.

In a further aspect of the invention, there is provided a method for detection of RSV B in a sample comprising the steps of obtaining a tissue sample from a patient; extracting nucleic acid from the sample; amplifying the nucleic acid, wherein the RNA is amplified and detected with amplification primers and detection probes of SEQ ID NOS. 7, 8, 9 and 10.

In still another aspect of the invention, there is provided a method for detection of hMPV in a sample comprising the steps of obtaining a tissue sample from a patient; extracting nucleic acid from the sample; amplifying the nucleic acid, wherein the RNA is amplified and detected with amplification primers and detection probes of SEQ ID NOS. 11, 12, 13 and 14.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

In a further embodiment of the invention, the assays may be conducted as a singleplex assay to detect one virus or in a multiplex assay to detect more than one virus. Where the assay is a multiplex assay, the multiplex assay may be used to identify viruses selected from the group consisting of RSV A, RSV B, hMPV, influenza A, influenza B, parainfluenza viruses, adenovirus, coronavirus, and rhinoviruses.

Additional aspects, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 is a bar graph showing the results of the Respiratory Panel assays described in Example 1.

### DETAILED DESCRIPTION OF THE INVENTION

Set forth below is a description of what are currently believed to be the preferred embodiments and best examples of the claimed invention. Any alternates or modifications in function, purpose, or structure are intended to be covered by the claims of this application.

In describing and claiming the present invention, the following terminology and the following definitions are used for the purpose of describing particular embodiments only, and are not intended to be limiting. As used in this specification and the appended claims, the singular forms “a,” “an,” and “the” include plural referents unless the context clearly dictates otherwise.

The term “singleplex” refers to a single assay that is not carried out simultaneously with any other assays. Singleplex assays include individual assays that are carried out sequentially. Within the context of the present invention, a singleplex assay would be for example, a molecular assay that screens for only RSV or hMPV.

The term “multiplex” refers to multiple assays that are carried out simultaneously, in which detection and analysis steps are generally performed in parallel. As used herein, a multiplex assay may also be termed according to the number of target sites that the assay aims to identify. Within the context of the present invention, a multiplex assay would be for example, a molecular assay that simultaneously screens for RSV and hMPV, or RSV, hMPV, influenza A and influenza B.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide, whether produced naturally as in a purified restriction digest or produced synthetically, which is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “target amplification” refers to enzyme-mediated procedures that are capable of producing billions of copies of nucleic acid target. Examples of enzyme-mediated target amplification procedures known in the art include PCR, nucleic acid-sequence-based amplification (“NASBA”), transcription-mediated amplification (“TMA”), strand displacement amplification (“SDA”), and ligase chain reaction (“LCR”).

Within the context of the present invention, the nucleic acid “target” is the nucleic acid sequence of RSV A, RSV B, and hMPV.

The most widely used target amplification procedure is PCR, first described for the amplification of DNA by Mullins et al. in U.S. Pat. No. 4,683,195 and Mullis in U.S. Pat. No. 4,683,202. The PCR procedure is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.”

In the PCR technique, a sample of DNA is mixed in a solution with a molar excess of at least two oligonucleotide primers of 10-30 base pairs each that are prepared to be complementary to the 3′ end of each strand of the DNA duplex; a molar excess of unattached nucleotide bases (i.e., dNTPs); and DNA polymerase, (preferably Taq polymerase, which is stable to heat), which catalyzes the formation of DNA from the oligonucleotide primers and dNTPs. Of the primers, at least one is a forward primer that will bind in the 5′-3′ direction to the 3′ end of one strand of the denatured DNA analyte and another is a reverse primer that will bind in the 3′-5′ direction to the 5′ end of the other strand of the denatured DNA analyte. The solution is heated to 94-96° C. to denature the double-stranded DNA to single-stranded DNA. When the solution cools, the primers bind to the separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. In other words, an extension product synthesized from the forward primer, upon separation, would serve as a template for a complementary extension product synthesized from the reverse primer. Similarly, the extension product synthesized from the reverse primer, upon separation, would serve as a template for a complementary extension product synthesized from the forward primer. In this way, the region of DNA between the primers is selectively replicated with each repetition of the process. Since the sequence being amplified doubles after each cycle, a theoretical amplification of one billion copies may be attained after repeating the process for a few hours; accordingly, extremely small quantities of DNA may be amplified using PCR in a relatively short period of time.

The terms “amplification sequence” and “amplification product” are used interchangeably to refer to the single-stranded sequences that are the end product of a PCR.

Where the starting material for the PCR reaction is RNA, complementary DNA (“cDNA”) is made from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. The enzymes are used in genetic engineering to produce specific cDNA molecules from purified preparations of mRNA. A PCR used to amplify RNA products is referred to as reverse transcriptase PCR or “RT-PCR.”

The terms “kinetic PCR” (“kPCR”) or “kinetic RT-PCR” (“kRT-PCR”), which are also referred to as “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes used in kPCR and kRT-PCR include the following probes: TAQMAN® probes, Molecular Beacons probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quench molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green probes binds double-stranded DNA and upon excitation emit light; thus as PCR product accumulates, fluorescence increases.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer. Such minor modifications of the disclosed sequences and any necessary adjustments of hybridization conditions to maintain specificity require only routine experimentation and are within the ordinary skill in the art.

It is understood by one of ordinary skill in the art that the isolation of DNA and RNA target sequences from a sample requires different hybridization conditions. For example, if the sample is initially disrupted in an alkaline buffer, double stranded DNA is denatured and RNA is destroyed. By contrast, if the sample is harvested in a neutral buffer with SDS and proteinase K, DNA remains double stranded and cannot hybridize with the primers and/or probes and the RNA is protected from degradation.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation. Typical hybridizing conditions include the use of solutions buffered to a pH from about 7 to about 8.5 and temperatures of from about 30° C. to about 60° C., preferably from about 37° C. to about 55° C. for a time period of from about one second to about one day, preferably from about 15 minutes to about 16 hours, and most preferably from about 15 minutes to about three hours. Hybridization conditions also include an buffer that is compatible, i.e., chemically inert, with respect to primers, probes, and other components, yet still allows for hybridization between complementary base pairs, can be used. The selection of such buffers is within the knowledge of one of ordinary skill in the art.

The terms “support” and “substrate” are used interchangeably to refer to any solid or semi-solid surface to which an oligonucleotide probe or primer, analyte molecule, or other chemical entity may be anchored. Suitable support materials include, but are not limited to, supports that are typically used for solid phase chemical synthesis such as polymeric materials and plastics for use in beads, sheets, and microtiter wells or plates examples including without limitation, polystyrene, polystyrene latex, polyvinyl chloride, polyvinylidene fluoride, polyvinyl acetate, polyvinyl pyrrolidone, polyacrylonitrile, polyacrylamide, polymethyl methacrylate, polytetrafluoroethylene, polyethylene, polypropylene, polycarbonate, and divinylbenzene styrene-based polymers; polymer gels; agaroses such as SEPHAROSE®; dextrans such as SEPHADEX®); celluloses such as nitrocellulose; cellulosic polymers; polysaccharides; silica and silica-based materials; glass (particularly controlled pore glass) and functionalized glasses; ceramics, and metals. Preferred supports are solid substrates in the form of beads or particles, including microspheres, nanospheres, microparticles, and nanoparticles.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms (particularly ³²P and ¹²⁵I), electron-dense reagents, enzymes, and ligands having specific binding partners. Enzymes are typically detected by their activity. Within the context of flow cytometric analysis, preferred labels include biotinylated primary agents (such as biotinylated dNTPs) that hybridized to a target (such as an amplification sequence from a PCR) and streptavidin-phycoerythrin (“SA-PE”) as secondary agents, where the streptavidin acts as a developer by binding to the biotinylated primary agent and the phycoerythrin acts as the stain.

As used herein, the term “sample” as used in its broadest sense to refer to any biological sample from any human or veterinary subject that may be tested for the presence of one or more diseases, such as RSV A, RSV B, hMPV, influenza A, influenza B, parainfluenza viruses, adenovirus, coronavirus, and rhinoviruses. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The amplification primers and detection probes of the present invention are set forth in Examples 4 and 5. Example 4 (Table 6) sets forth the sequences for detection of RSV A (SEQ ID NOS. 1-3) and RSV B (SEQ ID NOS. 4-10). Example 5 (Table 7) sets forth the sequences for detection of hMPV (SEQ ID NOS. 11-14).

In one aspect of the invention, there is provided a method for detection of RSV A in a sample comprising the steps of obtaining a tissue sample from a patient; extracting nucleic acid from the sample; amplifying the nucleic acid, wherein the nucleic acid is amplified and detected with amplification primers and detection probes of SEQ ID NOS. 1, 2 and 3.

In another aspect of the invention, there is provided a method for detection of RSV B in a sample comprising the steps of obtaining a tissue sample from a patient; extracting nucleic acid from the sample; amplifying the nucleic acid, wherein the RNA is amplified and detected with amplification primers and detection probes of SEQ ID NOS. 4, 5 and 6.

In a further aspect of the invention, there is provided a method for detection of RSV B in a sample comprising the steps of obtaining a tissue sample from a patient; extracting nucleic acid from the sample; amplifying the nucleic acid, wherein the RNA is amplified and detected with amplification primers and detection probes of SEQ ID NOS. 7, 8, 9 and 10.

In still another aspect of the invention, there is provided a method for detection of hMPV in a sample comprising the steps of obtaining a tissue sample from a patient; extracting nucleic acid from the sample; amplifying the nucleic acid, wherein the RNA is amplified and detected with amplification primers and detection probes of SEQ ID NOS. 11, 12, 13 and 14.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

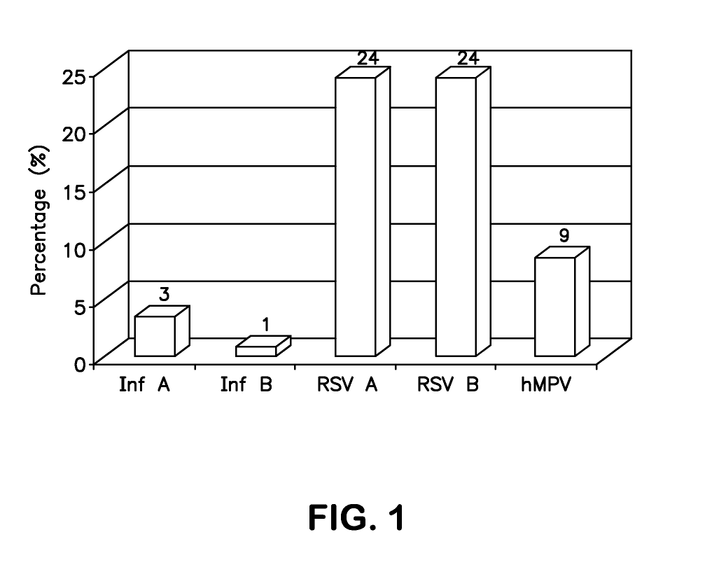

In a further embodiment of the invention, the assays may be conducted as a singleplex assay to detect one virus or in a multiplex assay to detect more than one virus. Where the assay is a multiplex assay, the multiplex assay may be used to identify viruses selected from the group consisting of RSV A, RSV B, hMPV, influenza A, influenza B, parainfluenza viruses, adenovirus, coronavirus, and rhinoviruses. For example, the primer and probe sets set forth in Examples 4 and 5 may be used in a real time RT-PCR assay in singleplex format to independently detect RSV A, RSV B, and hMPV, respectively, or in a multiplex format to simultaneously detect RSV A, RSV B, and hMPV.

Where a multiplex assay is used, the assay may designed to additionally include additional sequences such as the influenza A and B sequences that were used in Example 1 to run a multiplex assay that screens for RSV A, RSV B, hMPV, influenza A, and influenza B (FIG. 1). Table 1 of Example 2 shows that molecular assays of the present invention have increased sensitivity and accuracy over the rapid antigen assays presently used in the art. For the experiment described in Example 2, the data in Table 1 indicates that the RSV A/B assay described therein showed a 16% increase in RSV A/B detection over rapid antigen assays (19 additional samples/116 total samples=16%). Tables 2 to 5 in Example 3 show that increased accuracy of the RSV molecular assays of the present invention over the rapid antigen assays presently used in the art.

The RSV and hMPV assays described herein have utility in the field of clinical diagnostics. The RSV and hMPV assays, which are run on a kRT-PCR platform provide a sensitive and reliable method for detection of respiratory viruses and show potential for improved diagnostic yield over rapid antigen testing.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains.

All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXPERIMENTAL

The practice of the present invention will use, unless otherwise indicated, conventional techniques of molecular biology, biochemistry, microbiology, and the like, which are within the skill of the art. Such techniques are explained fully in the literature. See, for example, Sambrook, et al., MOLECULAR CLONING: A LABORATORY MANUAL, 2^{nd} ed. (1989); OLIGONUCLEOTIDE SYNTHESIS (M. J. Gait, ed., 1984); THE PRACTICE OF PEPTIDE SYNTHESIS (M. Bodanszky and A. Bodanszky, 2^{nd} ed., Springer-Verlag, New York, N.Y., 1994); NUCLEIC ACID HYBRIDIZATION (B. D. Haines & S. J. Higgins, eds., 1984); and METHODS IN ENZYMOLOGY (Elsevier, Inc., Burlington, Mass.).

In the examples that follow, efforts have been made to ensure accuracy with respect to numbers (e.g., amounts, temperature, etc.), but experimental error and deviations should be taken into account when conducting the described experiments. Unless indicated otherwise, parts are parts by weight, temperature is degrees centigrade and pressure is at or near atmospheric. All components were obtained commercially unless otherwise indicated.

In Tables 6 and 7 (Examples 3 and 4, respectively), “FP” indicates the forward primer and “RP” indicates the reverse primer. The sequences in the tables are identified as being either sense (complementary; “+”) or antisense (reverse complementary; “−”) to the coding strand.

The following protocols were used in the Examples.

Nasopharyngeal wash specimens were collected from 116 pediatric patients at the Henry Ford Hospital (Detroit, Mich.) from September 2005 to March 2006, that presented with moderate to severe respiratory symptoms (cough plus nasal congestion, fever, difficulty breathing). The patients ranged from 2 weeks to 9 years (average 13.5 months). Specimens were immediately tested for RSV antigen and residual specimen was stored for molecular testing.

RSV antigen detection was performed using the BD DIRECTIGEN® RSV assay (Becton, Dickinson and Co., Franklin Lakes, N.J.) if the specimen was submitted for stat testing or the BioRad PATHFINDER® RSV enzyme immunoassay (Bio-Rad Laboratories, Inc., Hercules, Calif.) if the specimen was submitted for routine testing.

The DIRECTIGEN® RSV assay is a calorimetric enzyme membrane-based immunoassay. Results are read visually with development of a purple color on the membrane indicating a positive test. An internal control appearing as a purple dot on the membrane demonstrates proper reagent function and correct test procedure. Total assay time is about 15 minutes per sample.

The PATHFINDER® RSV assay is a calorimetric enzyme tube-based immunoassay. Results are read spectrophotometrically with a cutoff value and calculated gray zone. Positive and negative controls are run with each assay. Total assay time is about 60 minutes per sample.

For the molecular assays (referred to as the “Bayer Research-Based RSV RT-PCR Assays,” the “Bayer RSV assay(s),” “Bayer RSV A,” “Bayer RSV B.” or “Bayer RSV A/B” assays as appropriate in the Examples that follow), RNA was extracted from a 200 μL aliquot of nasopharyngeal wash and purified using a QIAMP® MINELUTE® virus spin kit (Qiagen, Valencia Calif.).

Using the appropriate primers and probes, the RNA samples were screened for the presence of the viruses using real-time quantitative RT-PCR assays on the MX3000P® Real-Time PCT System (Stratagene Corp., La Jolla, Calif.), which analyzes fluorescence emitted during the annealing step within the amplification cycles. A run takes approximately 150 minutes for 90 samples.

Where volumes were sufficient, the Cepheid Analyte Specific Reagent (ASR) assay (Cepheid Corp., Sunnyvale, Calif.) was used to test for discrepant samples, i.e., samples that were rapid antigen negative, but RSV molecular positive. The Cepheid RSV ASR contains primers and a fluorescent labeled probe that is designed to detect total RSV without distinguishing between type A or B. It is an internally controlled assay that is performed using the Cepheid SMARTCYCLER®.

### Example 1

### Assay to Detect RSV A, RSV B, hMPV, Influenza A, and Influenza B in the 116 Pediatric Samples

An assay was performed to screen for RSV A, RSV B, influenza A, influenza B, and hMPV in a one-step, real-time quantitative RT-PCR assay. The amplicon size for the assays ranged from 82 base pairs (bp) to 202 bp. All assays had identical PCR cycling profiles. Each assay contained an internal control (IC) that was added during extraction to assess both the sample preparation and amplification processes. The IC consisted of a second heterologous amplification system and included primers, Taqman probes, and a unique target. As noted above, the Cepheid RSV ASR assay was independently performed to assess the discrepant specimens (RSV rapid antigen negative and Bayer RSV molecular positive). The results of the Respiratory panel assays are shown in FIG. 1. Of the 116 samples tested in the assay, RSV A and RSV B were detected in 28 samples each; influenza A was detected in 4 samples (3%); influenza B was detected in 1 sample (<1%); and hMPV was detected in 10 samples (9%).

### Example 2

### Rapid Antigen and Molecular Screening of Discrepant Samples

Comparative results of the rapid antigen and Bayer RSV molecular assays for the 116 pediatric samples is set forth in Table 1. As shown in the table, the rapid antigen assays detected RSV in 34 of the 116 samples. In the comparative experiment, the Bayer RSV assay accurately detected RSV A/B in 34 of 34 (100%) of the antigen positive samples. In addition to the foregoing, the Bayer assay also detected an additional 19 RSV A/B positive samples, all of which were confirmed via the Cepheid RSV ASR assay.

TABLE 1

BAYER RESEARCH-
BASED ASSAYS

| | | |
|---|---|---|
| N = 116 | + | − |

| | | | |
|---|---|---|---|
| RAPID | + | 34 | 0 |
| ANTIGEN | − | 19* | 62 |
| ASSAYS | INDETERMINATE | 1* | 0 |

*Samples independently tested using Cepheid RSV ASR

### Example 3

### Rapid Antigen and Molecular Screening of Discrepant Samples

The 20 discrepant samples from Example 1 (i.e., samples that were molecular assay positive, but rapid antigen negative) were screened using the BioRad PATHFINDER® and BD DIRECTPATH® rapid antigen assays and the Bayer RSV and Cepheid RSV ASR assays. The results of all assays are shown in Tables 2, 3, 4, and 5.

Of the 20 discrepant samples, the Bayer RSV assays found 8 of 20 samples (40%) to be RSV A positive; 10 of 20 samples (50%) to be RSV B positive; and 1 of 20 samples (5%) to be dually infected. The viral load for samples found to be positive using the Bayer RSV assays ranged from 29 to 2.8×10⁶ copies per reaction for RSV A and 16 to 6.6×10⁶ copies per reaction for RSV B.

The Bayer RSV assays and the Cepheid RSV ASR assay were concordant for 15 of 17 discrepant specimens (89%; of the 17 parallel samples tested with these two assay samples 3 and 4 tested positive with the Bayer RSV A assay and negative with the Cepheid RSV ASR assay).

The BioRad PATHFINDER® assay missed 8 of 15 RSV A samples (53%; specimens) and 7 of 15 RSV B samples (47%) and as shown in Tables 4 and 5; and the BD DIRECTIGEN® assay missed 5 of 5 RSV B specimens (100%).

TABLE 2

| DISCREPANT | BAYER | BIORAD | CEPHEID |
|---|---|---|---|
| SAMPLE NO. | RSV A | PATHFINDER ® | RSV ASR |
| 1 | 2,461,000 | − | * |
| 2 | 4,010,000 | − | + |
| 3 | 16 | − | − |
| 4 | 1,006,000 | − | − |
| 5 | 622,600 | − | + |
| 6 | 88 | − | + |
| 7 | 523,200 | − | + |
| 8 | 6,619,000 | − | * |

*Sample volume insufficient to allow testing with Cepheid RSV ASR.

TABLE 3

| DISCREPANT | BAYER | BIORAD | CEPHEID |
|---|---|---|---|
| SAMPLE NO. | RSV B | PATHFINDER ® | RSV ASR |
| 9 | 9,411 | − | + |
| 10 | 123,600 | Indeterminate | + |
| 11 | 2,286 | − | + |
| 12 | 239 | − | + |
| 13 | 21,070 | − | + |
| 14 | 29 | − | + |
| 15 | 95 | − | * |

TABLE 4

| DISCREPANT | BAYER | BD | CEPHEID |
|---|---|---|---|
| SAMPLE NO. | RSV B | DIRECTIGEN ® | RSV ASR |
| 16 | 2,757,000 | − | + |
| 17 | 1,096,000 | − | + |
| 18 | 2,310 | − | + |
| 19 | 11,580 | − | + |

TABLE 5

| DISCREPANT | BAYER | BD | CEPHEID |
|---|---|---|---|
| SAMPLE NO. | RSV A/B | DIRECTIGEN ® | RSV ASR |
| 20 | 10,830/84,190 | − | + |

### Example 4

### Identification of RSV Sequences

Table 6 identifies amplification primer sequences and detection probe sequences for detection of RSV A and RSV B. The highly conserved primer and probe sets set forth in the table were designed in the fusion protein gent. The map location references Accession Number L25351 for RSV A and Accession Number D00334 for RSV B. The primers and probes were designed based on alignment of 7 RSV A and 12 RSV B sequences.

TABLE 6

| | MAP | | |
|---|---|---|---|
| SEQ NAME | LOCATION | SEQUENCES 5′-3′ | STRAND |
| RSV-A-FP- | 1232-1254 | TCTAGGAGCCATTGTGTCATGCT | (+) |
| fwd | | (SEQ ID NO. 1) | |
| RSV-A-FP- | 1326-1299 | AATCRCACCCGTTAGAAAATGTCTTTAT | (−) |
| rev | | (SEQ ID NO. 2) | |
| RSV-A-FP- | 1256-1283 | R-TGGCAAAACTAAATGTACAGCATCCAAT-Q | (+) |
| probe | | (SEQ ID NO. 3) | |
| RSV-B-FP- | 803-828 | ATGCCTATAACAAATGACCAGAAAAA | (+) |
| fwd (set 1) | | (SEQ ID NO. 4) | |
| RSV-B-FP- | 912-885 | ACATATGCAAGGACTTCTTCCTTTATTA | (−) |
| rev (set 1) | | (SEQ ID NO. 5) | |
| RSV-B-FP- | 834-873 | R-TGTCAAGCAATGTTCAGATAGTAAGGCAACAAAGTTATTC-Q | (+) |
| probe (set 1) | | (SEQ ID NO. 6) | |
| RSV-B-FP- | 905-933 | GCATATGTTGTACAGCTACCTATCTATGG | (+) |
| fwd (set 2) | | (SEQ ID NO. 7) | |
| RSV-B-FP- | 1033-1004 | TCCTCTATCAGTCCTTGTTAAACAAATATT | (−) |
| rev1 (set 2) | | (SEQ ID NO. 8) | |
| RSV-B-FP- | 1043-1015 | CACARTACCATCCTCTATCAGTCCTTGT | (−) |
| rev2 (set 2) | | (SEQ ID NO. 9) | |
| RSV-B-FP- | 950-980 | R-TGCTGGAAATTACACACATCACCTCTATGCA-Q | (+) |
| probe (set 2) | | (SEQ ID NO. 10) | |

### Example 5

### Identification of hMPV Sequences

Table 7 identifies the amplification primer sequences and detection probe sequences for detection of hMPV. The highly conserved primer and probe sets set forth in the table were designed in the fusion protein gent. The map location references Accession Number AY304360. The primers and probes were designed based on alignment of 21 human hMPV sequences.

TABLE 7

| | MAP | | |
|---|---|---|---|
| SEQ NAME | LOCATION | SEQUENCES 5′-3′ | STRAND |
| hMPV-FP-fwd | 574-598 | AGCTTCAGTCAATTCAACAGAA | (+) |
| | | GRT (SEQ ID NO. 11) | |
| hMPV-FP-rev1 | 662-637 | AGGTCCAATGATATTGCTGGTG | (−) |
| | | TTAT (SEQ ID NO. 12) | |
| hMPV-FP-rev2 | 662-635 | AAGTCCAAAGATATTGCTGGTG | (1) |
| | | TTATTC (SEQ ID NO. 13) | |
| hMPV-FP-probe | 609-632 | R-TGTGCGGCARTTTTCAGACA | (+) |
| | | ATGC-Q (SEQ ID NO. 14) | |

1. a method for detection of respiratory syncytial virus type A (RSV: A) in a sample comprising the steps of:
obtaining a sample from a patient;
extracting nucleic acid from the sample;
amplifying the nucleic acid,
wherein the nucleic acid is amplified and detected with amplification primers and detection probes selected from the group consisting of SEQ ID NOS. 1, 2, and 3.

2. A method for detection of respiratory syncytial virus B (RSV B) in a sample comprising the steps of:
obtaining a sample from a patient;
extracting nucleic acid from the sample;
amplifying the nucleic acid,
wherein the RNA is amplified and detected with amplification primers and detection probes selected from the group consisting of SEQ ID NOS. 4, 5, and 6.

3. A method for detection of respiratory syncytial virus B (RSV B) in a sample comprising the steps of:
obtaining a sample from a patient;
extracting nucleic acid from the sample;
amplifying the nucleic acid,
wherein the RNA is amplified and detected with amplification primers and detection probes selected from the group consisting of SEQ ID NOS. 7, 8, 9, and 10.

4. A method for detection of human metapneumovirus (hMPV) in a sample comprising the steps of:
obtaining a sample from a patient;
extracting nucleic acid from the sample;
amplifying the nucleic acid,
wherein the RNA is amplified and detected with amplification primers and detection probes selected from the group consisting of SEQ ID NOS. 11, 12, 13, and 14.

5. The method of claim 1, wherein the nucleic acid is selected from RNA and DNA.

6. The method of claim 5, wherein the RNA is amplified using real time RT-PCR.

7. The method of claim 5, wherein the DNA is amplified using real time PCR.

8. The method of claim 1, wherein the sample is a fluid.

9. The method of claim 8, wherein the fluid is selected from the group consisting of blood, plasma, serum, lymphatic fluid, synovial fluid, cerebrospinal fluid, amniotic fluid, amniotic cord blood, tears, saliva, and nasopharyngeal washes.

10. The method of claim 1, wherein the method is a singleplex assay used to identify a single virus.

11. The method of claim 1, wherein the method is a multiplex assay used to identify at least one additional virus.

12. The method of claim 11, wherein the at least one additional virus is selected from the group consisting of respiratory syncytial virus B, human metapneumovirus, influenza A, influenza B, parainfluenza viruses, adenovirus, coronavirus, and rhinoviruses.

13. The method of claim 2, wherein the method is a multiplex assay used to identify at least one additional virus.

14. The method of claim 13, wherein the at least one additional virus is selected from the group consisting of respiratory syncytial virus A, human metapneumovirus, influenza A, influenza B, parainfluenza viruses, adenovirus, coronavirus, and rhinoviruses.

15. The method of claim 4, wherein the method is a multiplex assay used to identify at least one additional virus.

16. The method of claim 15, wherein the at least one additional virus is selected from the group consisting of respiratory syncytial virus A, respiratory syncytial virus B, influenza A, influenza B, parainfluenza viruses, adenovirus, coronavirus, and rhinoviruses.
